(19) United States
(12) Patent Application Publication
(10) Pub. No.: US 2013/0053630 A1
(43) Pub. Date: Feb. 28, 2013

(54) SMART CAPSULE FOR PROVIDING SEXUAL PLEASURE

(71) Applicants: Shay WAIL, Katzir (IL); Chananiel WEINRAUB, Ramat Hashron (IL)

(72) Inventors: Shay WAIL, Katzir (IL); Chananiel WEINRAUB, Ramat Hashron (IL)

(21) Appl. No.: 13/217,299

(22) Filed: Aug. 25, 2011

(51) Int. Cl. A61F 5/00; A61H 1/00

(52) U.S. Cl. 600/38; 601/46; 601/15

(57) ABSTRACT

A device for enhancing sexual pleasure includes an appliance, a memory for storing code for operating the appliance, and a processor for executing the code, all housed in a housing configured for insertion, as such or inside a condom, into a sexual organ. The appliance could be a stimulator or a sensor. The stimulator could be a speaker, a heater, a thermoelectric cooler, a source of electromagnetic radiation such as visible light, a source of electrical power or a mechanism for pressurizing a pressurizable medium. The sensor could be a thermometer, a microphone, a camera or an accelerometer.

### FIELD AND BACKGROUND OF THE INVENTION

The present invention relates to a device for enhancing sexual pleasure, and, more particularly, to such a device that is controlled by an on-board or remote computer.

Kettles et al., in US Patent Application Publication No. 2010/025052, teaches a condom with a pocket for inserting a contoured vibrator. US 2010/025052 is incorporated by reference for all purposes as if fully set forth herein.

Cool & Groovy Toy Company Ltd. of Lutterworth UK offers for sale a sexual appliance they call TheToy™. TheToy™ is a vibrator, for insertion in a woman's vagina, that is controlled remotely by her lover. Specifically, her lover uses his mobile phone to send SMS messages to TheToy™.

### SUMMARY OF THE INVENTION

The present invention combines and enhances aspects of these two prior art technologies to provide innovative modes of enhanced sexual pleasure. The enhanced pleasure could be direct enhancement, provided directly by a device of the present invention itself, or indirect enhancement, for example following diagnosis of sexual malfunction or therapy for sexual malfunction using the device.

According to the present invention there is provided a device for enhancing sexual pleasure, including: (a) a housing configured for insertion in a sexual organ of a user; and (b) within the housing: (i) an appliance selected from the group consisting of a stimulator and a sensor, wherein the stimulator is selected from the group consisting of: (A) a speaker, (B) a heater, (C) a thermoelectric cooler, (D) a source of electromagnetic radiation, (E) a source of electrical power, and (F) a mechanism for pressurizing a pressurizable medium, (ii) a memory for storing code for operating the appliance, and (iii) a processor for executing the code.

According to the present invention there is provided a computer-readable storage medium having computer-readable code embodied in the computer-readable storage medium, the computer-readable code including program code for operating an appliance, of a device for enhancing sexual pleasure, selected from the group consisting of a stimulator and a sensor, wherein the stimulator is selected from the group consisting of a speaker, a heater, a thermoelectric cooler, a source of electromagnetic radiation, a source of electrical power and a mechanism for pressurizing a pressurizable medium.

The device of the present invention is a device for enhancing sexual pleasure. The enhancement could be direct, as with embodiments of the device that are sex toys, or indirect, as with embodiments of the device for diagnosing and treating sexual dysfunction.

The device includes a housing configured for insertion in a sexual organ of a user, typically the vagina (vaginal sex), the anus (anal sex) or the mouth (oral sex). The housing could be configured, like TheToy™, to be inserted as such, or could be configured to be inserted in a condom. For example, the housing could be configured to be inserted in a pocket of a condom, similar to the condom of Kettle et al., that one user wears on his penis for insertion in the other user's sexual organ; or the housing could be configured to be covered by a conventional condom as the user rolls the condom onto his erect penis.

Inside the housing there are one or more appliances, a memory for storing code for operating the appliance(s), and a processor for executing the code. The appliances could be stimulators and/or sensors. Examples of stimulators include speakers, heaters (resistance or infrared), thermoelectric coolers, sources of electromagnetic radiation such as visible light and infrared light, sources of electrical power (electrical voltage and/or electrical current), and mechanisms for pressurizing a pressurizable medium. If one or more of the stimulators is a source of visible light then optionally the device also includes one or more flexible optical pipes that are optically coupled to the light source(s) and that extend at least partially outside the housing. If one or more of the stimulators is a pressurizing mechanism then the device also includes one or more flexible conduits, of the pressurizable medium, that operationally coupled to the mechanism(s) and that extend at least partially outside the housing. One of the stimulators could be a vibrator, but only together with other appliances (typically together with a speaker, so the vibrator can be made to vibrate in time to audible sounds such as music), because sex toys with just vibrators are known in the art. Examples of sensors include thermometers, microphones, cameras and accelerometers. Possible functionality of the code stored in the memory includes providing stimuli to the stimulators and recording measurements acquired by the sensors.

Preferably, if one or more of the appliances is a sensor, there also is, inside the housing, a transmitter for transmitting measurements acquired by the sensor(s). Also preferably, if one or more of the appliances is a stimulator, there also is, inside the housing, a receiver for receiving commands for operating the stimulator(s).

The scope of the present invention also includes a computer-readable storage medium on which is embodied program code for operating one or more of the appliances. If the appliance is a stimulator, the code preferably includes code (e.g. for a device of the present invention) for receiving commands for operating the stimulator or code (e.g. for a remote control) for transmitting the commands. If the appliance is a sensor, the code preferably includes code (e.g. for a device of the present invention) for transmitting measurements acquired by the sensor or code (e.g. for a remote control) for receiving measurements acquired by the sensor.

### BRIEF DESCRIPTION OF THE DRAWINGS

Various embodiments are herein described, by way of example only, with reference to the accompanying drawings, wherein:

FIG. 1 is a partial schematic block diagram of a device of the present invention;

FIG. 2 is a partial schematic block diagram of a remote control for use with the device of FIG. 1;

FIG. 3 is a cross section of one exemplary device of the present invention;

FIG. 4 is an exterior view of another exemplary device of the present invention.

### DESCRIPTION OF THE PREFERRED EMBODIMENTS

The principles and operation of a device for enhancing sexual pleasure according to the present invention may be better understood with reference to the drawings and the accompanying description.

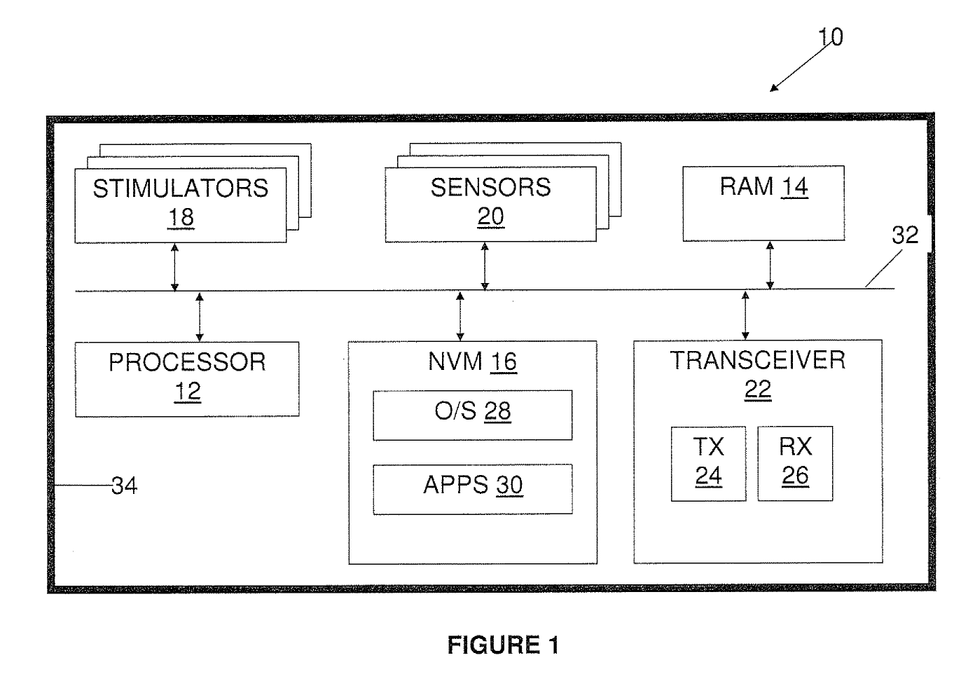

Referring now to the drawings, FIG. 1 is a partial schematic block diagram of a device 10 of the present invention. Device 10 includes a processor 12, a random access memory (RAM) 14, a non-volatile memory (NVM) 16 that typically is a flash memory, one or more stimulators 18 and/or one or more sensors 20, and a wireless transceiver 22 that includes a wireless transmitter 24 and/or a wireless receiver 26, all within a housing 34. Components 12, 14, 16, 18, 20 and 22 communicate with each other via a common bus 32. (In other embodiments, components 14, 16, 18, 20 and 22 are connected directly to processor 12.) In NVM 16 is stored executable code for operating device 10: an operating system 28 and application code 30 for operating whichever stimulators 18 and/or sensors 20 are installed in device 10 and, optionally, for operating transceiver 22 in conjunction with appliances 18 and/or 20.

FIG. 1 is a partial illustration in the sense that required and optional components that are not needed to explain the principles of the present invention are not illustrated. Such components include a power supply and a read-only memory for storing boot code that processor 12 executes to load operating system code 28 from NVM 16 into RAM 14. Processor 12 executes operating system code 28 in RAM 14 to operate device 10. Under the control of operating system code 28, processor 12 loads application, code 30 into RAM 14 and executes application code 30 in RAM 14 to operate whichever stimulators 18 and/or sensors 20 are installed in device 10.

Stimulators 18 and sensors 20 are optional in the sense that device 10 must include either at least one stimulator 18 or at least one sensor 20 but need not include both stimulators and sensors. Specific examples of stimulators 18 and sensors 20 are described below.

Housing 34 is configured to be inserted comfortably in a sexual organ of a user. One typical configuration of housing 34 is in the size and shape of a vibrator of Kettles et al. Such a device 10 could be inserted in a pocket of a condom of Kettles et al. or could be held in place by the elasticity of a conventional condom that is rolled onto a user's erect penis in the conventional manner. Another typical configuration of housing 34 is in the size and shape of TheToy™. Typical dimensions of a device 10 intended for insertion in a pocket of a condom are 24-25 mm length, 12-15 mm width, 5-6.5 mm height.

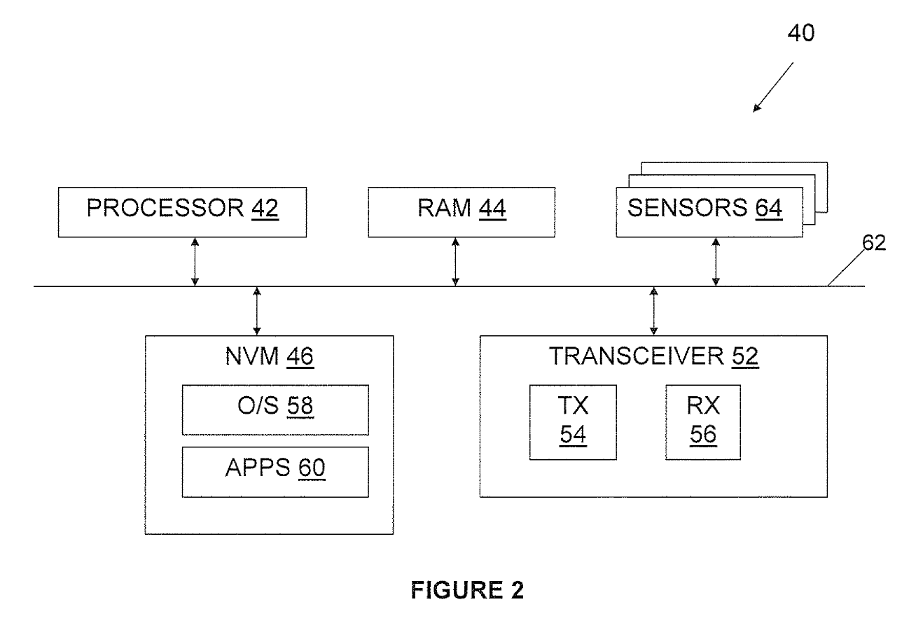

FIG. 2 is a partial schematic block diagram of a remote control 40 for operating device 10 in a manner similar to that in which the lover of the user of TheToy™ operates TheToy™ with his mobile phone. Remote control 40 includes a processor 42, a RAM 44, a NVM 46, one or more sensors 64 and a wireless transceiver 52 that includes a wireless transmitter 54 and/or a wireless receiver 56, all communicating with each other via a common bus 62. (In other embodiments, components 44, 46, 64 and 52 are connected directly to processor 42.) In NVM 46 is stored executable code for operating remote control 40: an operating system 58 and application code 60 for remotely operating one or more of the stimulators 18 and/or sensors 20 that are installed in device 10 and for operating transceiver 52 in conjunction with appliances 18 and/or 20. Note that, depending on the nature of the relevant stimulator 18 or sensor 20, application code 30 may be sufficient by itself to operate that stimulator 18 or sensor 20, or application codes 30 and 60 may cooperate in operating that stimulator 18 or sensor 20.

Sensors 64 are optional, and may include an accelerometer, a microphone, a camera, and/or a thermometer. The accelerometer, microphone and camera would be for receiving user inputs that control device 10: the accelerometer via measurements of the movement, tilting, and/or rolling of remote control 40; the microphone via voice commands; and the camera via gestures and facial expressions. The thermometer helps in controlling the thermoelectric cooler of device 10 described below or the heater of device 10 described below relative to ambient temperature.

As in the case of FIG. 1, FIG. 2 is a partial illustration in the sense that components that are not needed to illustrate the principles of the present invention, such as a power supply and a read-only memory for storing boot code, are not shown in FIG. 2.

Receiver 26 is obligatory only if device 10 includes a stimulator 18 that needs to be operated in cooperation with remote control 40. In such a case, transmitter 54 also is obligatory. Similarly, transmitter 24 is obligatory only if device 10 includes a sensor that needs to be operated in cooperation with remote control 40. In such a case, receiver 56 also is obligatory. Transceivers 22 and 52 are conventional wireless transceivers, for example Bluetooth® transceivers. Note that the Bluetooth® protocol may require each of transceivers 22 and 52 to include both a transmitter and a receiver.

It is not accidental that FIGS. 1 and 2 resemble block diagrams of generic computers. Device 10 and remote control 40 are indeed computers that are configured for enhancing sexual pleasure and performance. Remote control 40 could very well be a conventional laptop computer, in which case operating system 58 typically is a member of the Windows™ family of operating systems. Typically, however, remote control 40 is a mobile device such as a smartphone, and operating system 58 could be, for example, the Android™ operating system.

NVMs 16 and 46 are examples of computer-readable storage media having embodied thereon computer-readable code of the present invention. Other examples of such media include flash disks and compact disks.

Several examples of appliances (stimulators 18 and sensors 20) of the present invention now will be described. The theme of this collection of appliances is “different strokes for different folks”.

Stimulators 18

Speaker: A speaker is used to play music that is stored in NVM 16 or that is stored in NVM 46 and transmitted by remote control 40 to device 10. If one of stimulators 18 is a vibrator, the vibrator can be operated rhythmically in time with the music. The vibrator could be implemented as a circular motor, as a disk motor, as an elastic diaphragm or as a piezoelectric membrane. A suitably configured speaker could do double duty as a vibrator.

Thermoelectric cooler: A Peltier effect device establishes a temperature difference between two sides of device 10. Such a thermoelectric cooler is most useful in a device 10 that is intended for insertion in a pocket of a condom. The temperature difference across the thermoelectric cooler can be e.g. up to 10° C. The actual temperature difference experienced by the users is less than 10° C. because the temperature difference is moderated by the components of device 10 that surround the thermoelectric cooler. Application code 30 and/or 60 allows users to adjust the temperature difference to whatever temperature difference they find comfortable and enjoyable. The cooled side of device 10 can be either the side facing the penis in the condom or the side facing the orifice outside the condom.

Heater: A resistance heater or an infrared heater (a source of infrared light)) can be used in the same manner as the thermoelectric cooler, with the difference that both sides of device 10 are heated instead of one side being heated while the other side is cooled. In a device 10 configured like TheToy™, such a heater also has therapeutic applications, for example for treating vaginal infections.

Electrical stimulator: Such a stimulator includes circuitry such as a charge pump voltage multiplier, electrically coupled to hypoallergenic electrodes on the top surface and/or the bottom surface of device 10 if device 10 is configured for insertion into a condom or into a pocket of a condom, or on a lateral surface of device 10 if device 10 is configured like TheToy™. In the case of a device 10 that is configured for insertion into a condom or into a pocket of a condom, the condom should have electrically conductive material adjacent to the top and/or bottom surface electrodes, or alternatively one or two slits on its top to expose the top surface electrodes. Safe ranges of current and voltage are, for example, respectively, 1 mA to 5 mA and 0.1 V to 5V. Application code 30 and/or 60 allows users to adjust the current and voltage to whatever levels they find comfortable and enjoyable for example to provide gentle itching or tickling. If one of stimulators 18 is a speaker, the electrical stimulator can deliver mild electrical shocks in time to music. In some embodiments the music is stored in NVM 16 and played by a speaker 18. In other embodiments, the music is played by remote control 40.

Electromagnetic radiation: A source of visible light, such as one or more LEDs, provides steady or blinking light in one or more colors. If one of stimulators 18 is a speaker, the light can blink in time with music. Optionally, the LEDs) is/are optically and mechanically coupled to flexible light pipes that protrude from device 10 and that spread the illumination while adding tactile variety to the general sexual pleasure provided by device 10. In a device 10 configured like TheToy™, a source of infrared light has therapeutic applications, for example for treating vaginal infections.

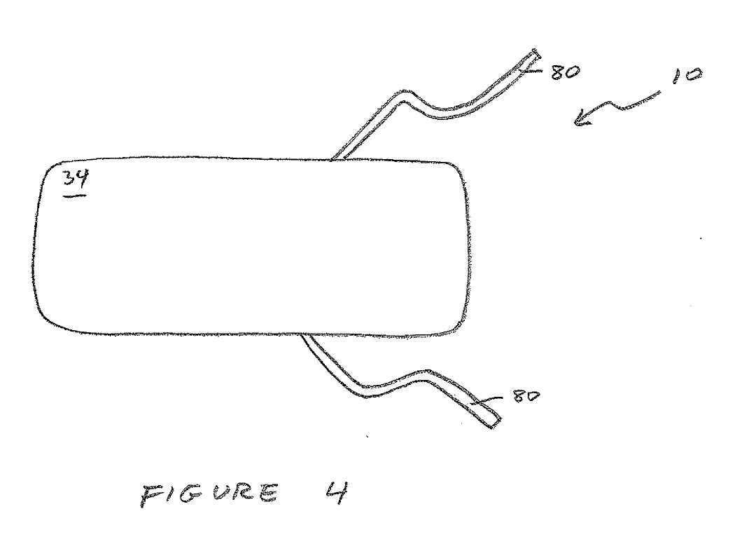

Pressurizing mechanism: This stimulator is discussed below in the context of FIG. 4.

Sensors 20

Thermometer: Feedback from a thermometer regulates the temperature difference imposed by a thermoelectric cooler or the temperature elevation imposed by a heater. Records of temperature during sexual intercourse are useful for medical diagnostic purposes.

Camera: In a device 10 configured for insertion in a condom or in a pocket of a condom, if the condom or the pocket has a suitable transparent portion or a suitable opening to expose the lens of a miniature camera, the camera is used to record a video image of the orifice surrounding the condom during sexual intercourse. These images are recorded in NVM 16 or in RAM 14 for later playback, and/or are transmitted to remote control 40 to be recorded in NVM 46 or in RAM 44 for later playback.

Microphone: In a device 10 configured for insertion in a condom or in a pocket of a condom, a microphone is used to record sounds within the orifice surrounding the condom during sexual intercourse. These sounds are recorded in NVM 16 or in RAM 14 for later playback or are transmitted to remote control 40 to be recorded in NVM 46 or in RAM 44 for later playback.

Accelerometer: Records of penile motion during sexual intercourse are useful for diagnosing a variety of medical conditions, including but not limited to Priapism and premature ejaculation. In addition, some users may be inclined to show off records of numbers of penetration, rate of penetration and penetration intensity, for bragging rights.

Records of measurements performed by a sensor 20 and of data obtained by a sensor 20 can be transmitted to remote control 40 in real time or can be recorded in NVM 16 or in RAM 14 for later transmission to remote control 40. A device 10 configured like TheToy™ also can be provided with a USB port for later downloading of such records.

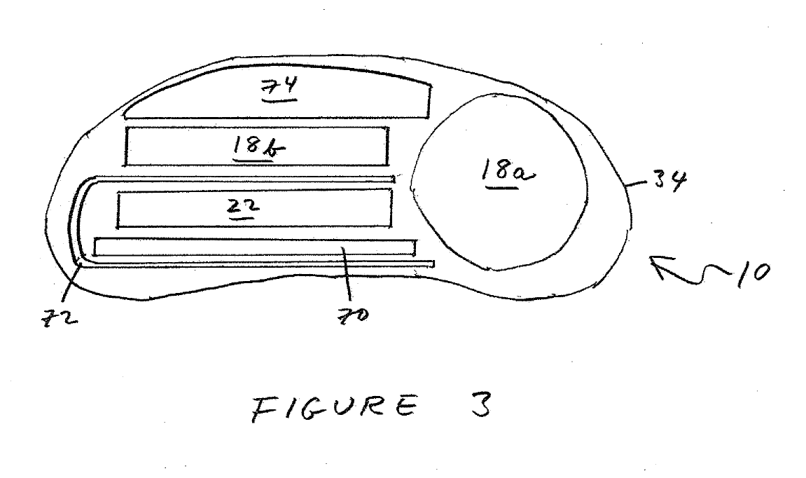

FIG. 3 is a cross-section through an embodiment of device 10 intended for insertion in a condom or in a pocket of a condom. This embodiment of device 10 includes two stimulators 18, a vibrator (circular motor) 18a and a thermoelectric cooler 18b. Electronic components other than transceiver 22 are mounted on a printed circuit board 70. If there otherwise would be a gap between thermoelectric cooler 18b and housing 34 then thermal contact between thermoelectric cooler 18b and housing 34 is provided by a thermal interface material 74 such as “Gap Filler 3500S35”™ available from The Bergquist Company of Chanhassen Minn. USA, and a metal foil 72. Metal foil 72 also may serve as the antenna of transceiver 22.

FIG. 4 is an exterior view of an embodiment of device 10 that includes two flexible optical pipes 80 that protrude from housing 34 and that are optically coupled to LEDs inside housing 34.

FIG. 4 also serves to illustrate an embodiment of device 10 in which stimulators 18 include a mechanism for pressurizing a pressurizable medium. In that context, reference numeral 80 refers to a flexible conduit that contains the pressurizable medium that is operationally coupled to the pressurizing mechanism and that protrudes from housing 34. Exemplary pressurizable media include electroactive polymers (either electronic electroactive polymers or ionic electroactive polymers) such as the electroactive polymers that are used in artificial muscles, a gas such as air, and a liquid such as water or a saline solution. If the pressurizable medium is an electroactive polymer then the pressurizing mechanism is a mechanism for reversibly applying an electric field to the electroactive polymer. If the pressurizable medium is a gas then the pressurizing mechanism is a pneumatic mechanism. If the pressurizable medium is a liquid then the pressurizing mechanism is a hydraulic mechanism. Typically, conduit(s) 80 is/are wrapped around housing 34 to provide pleasurable textural stimuli to the users.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A device for enhancing sexual pleasure, comprising:
(a) a housing configured for insertion in a sexual organ of a user; and
(b) within said housing:
(i) an appliance selected from the group consisting of a stimulator and a sensor, wherein said stimulator is selected from the group consisting of:
(A) a speaker,
(B) a heater,
(C) a thermoelectric cooler,
(D) a source of electromagnetic radiation,
(E) a source of electrical power, and
(F) a mechanism for pressurizing a pressurizable medium,
(ii) a memory for storing code for operating said appliance, and
(iii) a processor for executing said code.

2. The device of claim 1, wherein said housing is configured to be inserted as such in said sexual organ.

3. The device of claim 1, wherein said housing is configured for insertion in a condom that is used by a sexual partner of the user.

4. The device of claim 1, wherein said appliance includes a speaker.

5. The device of claim 4, wherein said appliance includes a vibrator, and wherein said code includes code for playing audible sounds via said speaker while operating said vibrator in synchrony with said audible sounds.

6. The device of claim 1, wherein said appliance includes a heater.

7. The device of claim 1, wherein said appliance includes a thermoelectric cooler.

8. The device of claim 1, wherein said appliance includes a source of electromagnetic radiation.

9. The device of claim 8, wherein said electromagnetic radiation includes visible light, the device further comprising:
(c) an optical pipe, optically coupled to said source of visible light and at least partly outside of said housing.

10. The device of claim 1, wherein said appliance includes a mechanism for pressurizing a pressurizable medium.

11. The device of claim 10, further comprising:
(c) a conduit of said pressurizable medium, operationally coupled to said mechanism and at least partly outside of said housing.

12. The device of claim 1, wherein said sensor is selected from the group consisting of:
(A) a thermometer,
(B) a microphone,
(C) a camera, and
(D) an accelerometer.

13. The device of claim 12, wherein said sensor includes a thermometer.

14. The device of claim 12, wherein said sensor includes a microphone.

15. The device of claim 12, wherein said sensor includes a camera.

16. The device of claim 12, wherein said sensor includes an accelerometr.

17. The device of claim 1, wherein said appliance includes said sensor, the device further comprising:
(c) within said housing, a transmitter for transmitting measurements acquired by said sensor.

18. The device of claim 1, wherein said appliance further includes said stimulator, the device further comprising:
(c) within said housing, a receiver for receiving commands for operating said appliance.

19. A computer-readable storage medium having computer-readable code embodied in the computer-readable storage medium, the computer-readable code comprising program code for operating an appliance, of a device for enhancing sexual pleasure, selected from the group consisting of a stimulator and a sensor, wherein the stimulator is selected from the group consisting of a speaker, a heater, a thermoelectric cooler, a source of electromagnetic radiation, a source of electrical power and a mechanism for pressurizing a pressurizable medium.

20. The computer-readable storage medium of claim 19, wherein said appliance includes said stimulator and wherein said program code includes program code for receiving commands for operating said stimulator.

21. The computer-readable storage medium of claim 19, wherein said appliance includes said sensor and wherein said program code includes program code for transmitting measurements acquired by said sensor.

22. The computer-readable storage medium of claim 19, wherein said appliance includes said stimulator and wherein said program code includes program code for transmitting commands for operating said stimulator.

23. The computer-readable storage medium of claim 19, wherein said appliance includes said sensor and wherein said program code includes program code for receiving measurements acquired by said sensor.

24. The computer-readable storage medium of claim 19, wherein the sensor is selected from the group consisting of a thermometer, a microphone, a camera and an accelerometer.
